(19) United States
(12) Patent Application Publication
(10) Pub. No.: US 2008/0241271 A1
(43) Pub. Date: Oct. 2, 2008

(54) Foaming anti-adhesion composition

(76) Inventors: Stephen B. Roman, Phoenix, AZ (US); Gary S. Kehoe, Phoenix, AZ (US)

Correspondence Address: TOD R NISSLE, PO BOX 55630, PHOENIX, AZ, 85078, US

(21) Appl. No.: 11/983,232

(22) Filed: Nov. 8, 2007

Related U.S. Application Data
(60) Provisional application No. 60/857,581, filed on Nov. 8, 2006.

(51) Int. Cl. A61K 33/30; A61P 31/12

(52) U.S. Cl. 424/641

(57) ABSTRACT

A method to treat nasal tissue reduces the ability of a virus to infect a human being. The method comprises the steps of providing a liquid composition comprising water, a humectant, a foam builder, 0.01% to 9% by weight of a surfactant, and a minor effective quantity of thickener to produce a coating on the nasal tissue after the liquid composition is applied to the nasal tissue as a foam; applying the liquid composition to the nasal tissue as a foam; and, allowing bubbles in the foam to dissipate so that a coating forms on the nasal tissue.

This application claims priority based on provisional patent application Ser. No. 60/857,581, filed Nov. 8, 2006.

This application pertains to methods and compositions to reduce the ability of a virus to infect a human being.

More particularly, the invention pertains to a composition which is applied as a foam to nasal tissues.

Nasal foams, although known, do not appear to be widely used. Such foams have been utilized to deliver medicaments that contact and penetrate nasal tissues.

There does not appear to be any market trend suggesting expanding the use of nasal foams. Indeed, just the opposite appears to be true. A review of commercially available compositions that are offered to consumers on store shelves and are applied to nasal tissue reveals sprays and other compositions and does not appear to reveal any compositions that are applied as foams. Assuming for sake of discussion that there are one or more commercially available nasal foams, it is likely that, as noted, there is no market trend toward expanding the use of foams, and further, it is likely that there is no problem with foams that is identified in the marketplace and that suggest a variety of predictable solutions. Since those of skill in the art have the foregoing as their knowledge of the art, there appears to be nothing in the marketplace associated with nasal foams that would fuel an investigation to produce an improved nasal foam, much less the unpredictable innovations set forth below in connection with the invention.

Accordingly, it is a principal object of the invention to provide an improved nasal foam.

We have discovered an improved composition to be applied to nasal tissues to reduce the ability of a virus to infect a human being. The composition is a liquid composition including water, a humectant, and, a surfactant. The liquid composition is applied to nasal tissue as a foam. When the liquid is applied in the nose as a foam, the foam tends to expand and cover a large portion of the nasal tissue that extends from the opening of the nose back toward the throat of a human being.

The nasal liquid composition can include a minor effective amount of a thickener/stabilizer to facilitate the formation of a gel coating on nasal membrane after the foam dissipates. In one preferred embodiment of the invention, the thickener includes a minor effective amount of iota and/or lambda carrageenan such that after the liquid nasal composition of the invention is applied as a foam and bubbles in the foam dissipate, a thixotropic gel forms on and coats the nasal tissues. Many thickener/stabilizers are known and can be utilized.

The nasal liquid composition can also include a foam builder that functions to extend the life of bubbles in the foam to facilitate expansion of the foam over tissue in the nasal cavity to permit the foam to coat more completely and evenly nasal membranes. Examples of foam builders are, without limitation, xanthan gum, myristylamine oxide, myristyl/cetyl amine oxide, sodium lauroyl sarcosinate, coconut diethanolamide, coco diethanolamide, lauryl lactyl lactate, anionic/nonionic blend (such as BIO-SOFT® LD-6 by the Stepan Company), sodium olefin sulfonate, PEG-5 cocamide, PEG-6 lauramide, and PEG-6 cocamide. The currently preferred foam builder in the nasal liquid composition of the invention is xanthan gum. If a foam builder is not included in the nasal liquid composition, then foam produced when the liquid nasal composition of the invention is dispensed in the nasal cavity may dissipate in a short period of time, possibly three seconds or less. When a foam builder is utilized, the foam produced typically will last at least four to twenty seconds in the nasal cavity, assuming that the user does not manipulate his nose to squeeze and break down the foam that is in the nasal cavity. The proportion of foam builder in the liquid nasal composition of the invention can be adjusted to extend the life of bubbles in foam to one minute, five minutes, or more. One advantage of the foam is that it tends to extend over and cover a large portion of the nasal membrane extending from the nostril openings back toward the throat of an individual.

As used herein, a foam builder is a composition that extends the life of a foam by strengthening bubbles comprising the foam such that at selected conditions (i.e., a defined temperature, atmospheric pressure, location, etc.) a longer period of time elapses before the bubbles break and the foam dissipates.

The preferred humectant(s) in the liquid nasal composition of the invention is glycerin. Alternate humectants are, by way of example and not limitation, propylene glycol (E1520), glyceryl triacetate (E1518), sorbitol (E420), xylitol and maltitol (E965), polymeric polyols like polydextrose (E1200), natural extracts like quillaia (E999), lactic acid, urea, and lithium chloride. The humectant is an important component of the invention because it facilitates the nasal composition maintaining an equilibrium RH and remaining moist after it is dispensed as a foam in the nasal cavity.

The presently preferred surfactant(s) in the liquid nasal composition of the invention is Crodasinic LS30 (TM) (30% sodium lauroyl sarcosinate sold by Croda, Inc.). Any desired surfactant can be utilized, but milder surfactants are preferred. By way of example, and not limitation, alternate surfactants include cocamidopropyl betaine, lauroyl sarcosine, cocoyl sarcosine, myristoyl sarcosine, oleoyl sarcosine, myristoyl/stearoyl sarcosine, sodium cocoyl sarcosinate, and sodium myristoyl sarcosinate. Mild anionic surfactants are preferred, although not required. The surfactant is a critical component of the liquid nasal composition of the invention because it is believed to interfere with the ability of a virus to infect the human body by entering the nasal cavity.

The pH of the nasal liquid composition can be adjusted by incorporating sodium hydroxide, citric acid, or other conventional pH adjusting components in the composition.

If desired, a preservative(s) such as benzalkonium chloride or benzyl alcohol can be incorporated in the nasal liquid composition.

The liquid nasal composition of the invention can be dispensed onto nasal membranes utilizing any desired apparatus that introduces air into the liquid composition to produce foam. Presently preferred is a container that permits a sequence of separate metered-doses to be delivered by the container while the container at the same time incorporates the correct amount of air into a dose while the dose is being delivered into the nasal cavity by the container. The size of the metered dose can be selected as desired, however, the presently preferred size of each dose is in the range of 0.05 to 0.95 gram, preferably 0.75 to 0.8 gram, and most preferably 0.1 to 0.6 gram. The container typically meters liquid nasal composition through a fine screen in the nozzle of the container while simultaneously incorporating air into the liquid composition. While the size of the bubbles in the foam dispensed by the nozzle of a container can vary as desired, the foam in large part presently preferably comprises small bubbles having a diameter of one centimeter (cm) or less, preferably a diameter of five mm or less, and most preferably a diameter of one mm or less. A quantity of foam comprised of small bubbles contains a greater percent by volume of the liquid nasal composition of the invention.

Carrageenan, in particular iota and/or lambda carrageenan, is an important component of the liquid nasal composition because it produces, after the foam dissipates, a thixotropic gel coating that covers and adheres to nasal membranes. The particular manner in which the thixotropic gel coating of the invention functions to interfere with the infection by the rhino virus or other virus of the human body is not definitely known, but it believed that the thixotropic gel prevents the virus from adhering to the ICAM-1, intracellular adhesion molecule, and ICAM-2 receptor sites in the nasal membrane or tissue in the nasal cavity.

Other auxiliary ingredients that can be included in the liquid nasal composition of the invention include green tea, zinc gluconate, zinc acetate, medicaments, polyols, acids, bases, extracts, aromatics, charged ions, minerals, vitamins, time-release drugs, natural decongestants such as eucalyptus oil and rosemary extract, and a cooling agent such a menthol. Although zinc, in the form of zinc gluconate or other acceptable zinc composition, can be incorporated in the nasal liquid composition of the invention, it is presently preferred not to utilize zinc and to therefore avoid the controversy surrounding the use of ionized metals in the nasal cavity.

One embodiment of the invention comprises a liquid nasal composition including 92.53% by weight water, 3.04% by weight humectant such a glycerin, 0.75% by weight surfactant such as cocamidopropyl betaine or Crodasinic LS30 (TM), 0.25% by weight foam builder such as zanthan gum, 1.50% by weight lambda carrageenan, 1.0% by weight iota carrageenan, 0.13% by weight of a pH adjuster such as sodium hydroxide, 0.05% by weight of a preservative such as benzalkonium chloride, and 1.0% by weight of a zinc composition such as zinc gluconate, although as noted it is presently preferred to use the liquid nasal composition of this paragraph by omitting the zinc.

Presently preferred weight percent ranges for components that can be utilized in the liquid nasal composition of the invention are 25% to 99.9% by weight water, 0.1% to 60% by weight humectant such as glycerin, 0.01% to 9% by weight surfactant such as cocamidopropyl betaine or Crodasinic LS30 (TM), 0.001% to 8% by weight foam builder such as zanthan gum, 0.01% to 25% by weight lambda and/or iota carrageenan, 0.01% to 2.5% by weight of a pH adjuster such as sodium hydroxide, 0.01% to 5.0% by weight of a preservative such as benzalkonium chloride, and, in the event zinc is utilized, 0.0001% to 8.0% by weight of zinc in the form of an acceptable zinc composition such as zinc gluconate.

Most preferred weight percent ranges for components that can be utilized in the liquid nasal composition of the invention are 85% to 99.9% by weight water, 1.5% to 6% by weight humectant such a glycerin, 0.25% to 1.25% by weight surfactant such as cocamidopropyl betaine or Crodasinic LS30 (TM), 0.1% to 1.1% by weight foam builder such as zanthan gum, 1.0% to 3.5% by weight lambda and/or iota carrageenan, 0.05% to 0.3% by weight of a pH adjuster such as sodium hydroxide, 0.01% to 0.2% by weight of a preservative such as benzalkonium chloride, and, in the event zinc is utilized, 0.0001% to 2.5% by weight of zinc in the form of an acceptable zinc composition such as zinc gluconate.

The water utilized may be de-ionized water, USP water, de-chlorinated water, mineral water, treated water, or tap water. The preferred water is de-ionized water.

If desired, 0.1% to 2.5% by weight kappa carrageenan can be utilized in combination with iota and/or lambda carrageenan. Some sources of carrageenan are, by way of example and not limitation, 373/Glecarin GP 111 (TM) (FMC), 335/Gelcarin GP 379 (TM) (FMC), and Genuvisco (TM) type X-923-03 (CP Kelco).

The following examples are provided by way of demonstration, and not limitation, of the invention.

### EXAMPLE I

The following ingredients are provided: 93.28% by weight water, 3.04% by weight glycerin, 0.75% by weight Crodasinic LS30 (TM), 0.25% by weight zanthan gum, 2.5% by weight iota carrageenan, 0.13% by weight of sodium hydroxide, and 0.05% by weight of benzalkonium chloride. The ingredients are combined as follows:
1. The water is weighed into a mixing vessel.
2. The glycerin (or other humectant) and the carrageenan are premixed. If zinc gluconate were utilized, it would also be premixed with the glycerin and carrageenan.
3. The glycerin and carrageenan mixture is mixed with the water in the mixing vessel.
4. The sodium hydroxide (or other pH adjusting composition) is added to the mixing vessel and admixed with the water, glycerin, carrageenan composition.
5. The Crodasinic LS30 (or other surfactant) is added to the mixing vessel and admixed with the water, glycerin, etc. composition.
6. The xanthan gum (or other foam building agent) is added to the mixing vessel and admixed with the water, glycerin etc. composition in the mixing vessel. Care is taken to not create a vortex during mixing so as to avoid aerating the product. Aeration is not desired in the final product.
7. The benzylalkonium chloride (or other preservative) is added to the mixing vessel and uniformly mixed with the other ingredients already in the mixing vessel.
8. Medicaments or other desired compositions can now, if desired, be added to the mixing vessel and uniformly mixed with the ingredients already in the mixing vessel.
9. The liquid mixture in the mixing vessel is allowed to cool slightly and is then loaded into dispensers of the type that dispense the liquid mixture as a foam.

### EXAMPLE II

A control group is selected comprising twenty-five healthy adults having ages ranging from twenty years old to fifty years old.

A first living area is selected. The area is cleaned and floors, counter tops, toilets, showers, and walls are substantially disinfected with bleach and other cleaning solutions. After the first living area is cleaned, the control group is isolated for two weeks in a selected area. Each member of the group bathes daily and washes his or her hands before and after meals, after using the restroom, and after touching dishes, clothes, or any objects brought into the selected area by outside individuals. Any physical contact with outside individuals is avoided during the two week period.

At the end of the two week period, none of the individuals in the control group appear to have contracted a cold or other illness and each appears healthy.

At the end of the two week period, the nasal membrane of each member of the control group is contacted with rhino virus that is on a cotton swab placed in each member's nose. Within three days of being contacted with the rhino virus, twenty-one members of the control group evidence symptoms of the common cold, i.e., running nose, watery eyes, etc.

A test group is selected comprising twenty-five healthy adults having ages ranging from twenty years old to fifty years old.

A second living area separate from the first living area is selected. The second living area is cleaned and floors, counter tops, toilets, showers, and walls are substantially disinfected with bleach and other cleaning solutions. After the second living area is cleaned, the test group is isolated for two weeks in a selected area. Each member of the test group bathes daily and washes his or her hands before and after meals, after using the rest room, and after touching dishes, clothes, or any objects brought into the selected area by outside individuals. Any physical contact with outside individuals is avoided during the two week period.

At the end of the two week period, none of the individuals in the test group appear to have contracted a cold or other illness and each appears healthy.

At the end of the two week period, each of the individuals is innoculated by applying in each nostril a 0.3 gm foam dose of the liquid composition of Example 1. The foam coats the nasal cavity and nasal membrane of each nostril and dissipated to form a gel coating on the nasal membrane. Several minutes after the foam doses are applied, rhino virus is placed in each nostril of each member of the test group by inserting a cotton swab containing the virus and contacting the gel coating. Within three days after each member of the test group is contacted by the rhino virus, only two members of the test group develop symptoms of the common cold, i.e., runny nose, watery eyes, etc.

### EXAMPLE III

The twenty-one members of the control group of EXAMPLE II that evidence symptoms of the common cold are treated by applying three times daily in each nostril a 0.3 gm foam dose of the liquid composition of EXAMPLE I. The time taken for eighteen of the twenty-one members to recover from the common cold symptoms is less than the normal recovery time associated with such common cold symptoms. Once the cold symptoms are gone, the daily treatments with foam doses are discontinued. The remaining three of the twenty-one members require a normal amount of time to recover from the common cold symptoms.

### EXAMPLE IV

Examples I to III are repeated, except that in Example 1, 1.0% by weight zinc gluconate is included and the amount of water is reduced from 93.28% to 92.28% by weight. Similar results are obtained.

### EXAMPLE V

Examples I to III are repeated, except that in Example 1, 1.5% by weight iota carrageenan is utilized instead of 2.5% by weight iota carrageenan, and the amount of water is increased from 93.28% to 94.28% by weight. Similar results are obtained.

### EXAMPLE VI

Examples I to III are repeated, except that in Example I, 2.5% by weight lambda carrageenan is utilized instead of iota carrageenan. Similar results are obtained.

### EXAMPLE VII

Examples I to III are repeated, except that in Example I, 0.5% by weight Crodasinic LS30 is utilized instead of 0.75% by weight, and the amount of water is increased from 93.28% to 93.53% by weight. Similar results are obtained.

### EXAMPLE VIII

Examples I to III are repeated, except that in Example I, 0.5% kappa carrageenan, 1.0% by weight lambda carrageenan, and 1.0% by weight iota carrageenan are utilized instead of 2.5% by weight iota carrageenan. Similar results are obtained.

### EXAMPLE IX

Examples I to III are repeated, except that in Example I, the amount of glycerin is 2.04% by weight instead of 3.04% by weight and the amount of water is increased from 93.28% to 94.28% by weight. Similar results are obtained.

### EXAMPLE X

Examples I to III are repeated, except that in Example 1, the amount of xanthan gum is 0.75% by weight instead of 0.25% by weight and the amount of water is decreased from 93.28% by weight to 92.78% by weight. Similar results are obtained.

### EXAMPLE XI

Examples I to III are repeated, except that in Example 1, the benzalkonium chloride and iota carrageenan are omitted and the amount of water is increased from 93.28% by weight to 95.83% by weight. Similar results are obtained.

### EXAMPLE XI

Examples I to III are repeated, except that in Example 1, the benzalkonium chloride, zanthan gum, and iota carrageenan are omitted and the amount of water is increased from 91.28% by weight to 96.08% by weight. Similar results are obtained.

### EXAMPLE XII

Examples I to III are repeated, except that in Example 1, the benzalkonium chloride, zanthan gum, sodium hydroxide, and iota carrageenan are omitted and the amount of water is increased from 91.28% by weight to 96.21% by weight. Similar results are obtained.

### EXAMPLE XIII

Examples I to III are repeated, except that in Example 1, the benzalkonium chloride, zanthan gum, sodium hydroxide, glycerin, and iota carrageenan are omitted and the amount of water is increased from 93.28% by weight to 99.25% by weight. Similar results are obtained.

### EXAMPLE XIV

Live rhino virus are placed in a petri dish. Ten milliliters of the liquid nasal solution of Example 1 are placed in the petri dish. The liquid nasal solution kills the rhino virus.

Having described the invention in terms sufficient to enable those of skill in the art to understand and practice the invention, we Claim:

1. A method to treat nasal tissue to reduce the ability of a virus to infect a human being, comprising the steps of
(a) providing a liquid composition comprising
(i) water,
(ii) a humectant,
(iii) a foam builder,
(iv) 0.01% to 9% by weight of a surfactant,
(v) a minor effective quantity of thickener to produce a coating on the nasal tissue after said liquid composition is applied to the nasal tissue as a foam;
(b) applying said liquid composition to the nasal tissue as a foam; and,
(c) allowing bubbles in said foam to dissipate so that a coating forms on the nasal tissue.

2. The method of claim 1 wherein said liquid composition includes 0.25% to 1.25% by weight of said surfactant.

3. The method of claim 1 wherein said liquid composition includes 1.5% to 6.0% by weight of said humectant.

4. The method of claim 1 wherein said liquid composition includes 0.0001% to 8.0% by weight zinc.
